(19) United States
(12) Patent Application Publication
(10) Pub. No.: US 2024/0198030 A1
(43) Pub. Date: Jun. 20, 2024

(54) CANNULA

(71) Applicant: NIHON KOHDEN CORPORATION, Tokyo (JP)

(72) Inventors: Hyo HIJIKATA, Tokorozawa-shi (JP); Yutaka UCHIYAMA, Tokorozawa-shi (JP); Toru MAEDA, Tokorozawa-shi (JP); Isao MATSUBARA, Tokorozawa-shi (JP); Ryosuke NAKANISHI, Tokorozawa-shi (JP)

(21) Appl. No.: 18/532,532

(22) Filed: Dec. 7, 2023

(30) Foreign Application Priority Data
Dec. 16, 2022 (JP) 2022-201397

(51) Int. Cl. A61M 16/06; A61M 16/08

(52) U.S. Cl. CPC A61M 16/0672; A61M 16/0683; A61M 16/0875; A61M 2202/0208

(57) ABSTRACT

The cannula includes an attachment portion configured to be attached in nasal cavities of a subject, and a junction portion configured to be connected to the attachment portion. A first connection portion is provided with a single medical gas flow path for supplying, to the subject, a medical gas from the medical gas supply device. The first connection portion being provided to the junction portion on a side of a medical gas supply device. A second connection portion is provided with a single measurement pipe for measuring a respiratory condition of the subject. The second connection portion being provided to the junction portion on a side of a respiration measurement module.

### CROSS-REFERENCE TO RELATED APPLICATIONS

This application is based on and claims priority under 35 USC 119 from Japanese Patent Application No. 2022-201397 filed on Dec. 16, 2022, the entire content of which is incorporated herein by reference.

### TECHNICAL FIELD

The present disclosure relates to a cannula.

### BACKGROUND ART

US2016/0296721A1 discloses a cannula with a bi-tubular interface having a gas supply conduit and a pressure conduit.

By the way, in a cannula used for high flow oxygen therapy, a high flow medical gas containing oxygen flows through a medical gas flow path provided in the cannula, and thus, a resistance applied to the medical gas flow path is relatively large. Therefore, for example, it is conceivable to provide a plurality of medical gas flow paths to reduce an amount of the medical gas flowing per one medical gas flow path. However, as the number of medical gas flow paths increases, the number of medical gas flow paths that come into contact with the outside air also increases, and thus, water droplets tend to be generated inside the medical gas flow paths through which a high-temperature and high-humidity medical gas flows. Furthermore, in the high flow oxygen therapy, it may be desirable to simultaneously supply a medical gas to a subject attached with a cannula and measure a respiratory condition of the subject. In this case, it is necessary to separately prepare a device for measuring the respiratory condition.

### SUMMARY OF INVENTION

Aspect of non-limiting embodiments of the present disclosure relates to provide a cannula having a simple configuration, and capable of making a medical gas flow path difficult to generate water droplets inside and of measuring a respiratory condition of a subject while supplying a high flow medical gas.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a cannula used in high flow oxygen therapy, the cannula including:
an attachment portion configured to be attached in nasal cavities of a subject; and
a junction portion configured to be connected to the attachment portion,
in which a first connection portion is provided with a single medical gas flow path for supplying, to the subject, a medical gas from the medical gas supply device, the first connection portion being provided to the junction portion on a side of a medical gas supply device, and
a second connection portion is provided with a single measurement pipe for measuring a respiratory condition of the subject, the second connection portion being provided to the junction portion on a side of a respiration measurement module.

### BRIEF DESCRIPTION OF DRAWINGS

Exemplary embodiment(s) of the present invention will be described in detail based on the following figures, wherein:

FIG. 1 is a schematic diagram of a medical system according to an embodiment of the present disclosure;

FIG. 2 is a perspective view illustrating a cannula according to a first embodiment;

FIG. 3 is a perspective view of the vicinity of an attachment portion and a junction portion of a cannula;

FIG. 4 is a vertical cross-sectional view of an internal structure of the attachment portion and the junction portion provided in the cannula according to the first embodiment;

FIG. 5 is a vertical cross-sectional view of the internal structure of the junction portion provided in the cannula according to the first embodiment;

FIG. 6 is a perspective view of the cannula in a state in which the junction portion is turned 180 degrees from a state illustrated in FIG. 2 and is mounted to the attachment portion;

FIG. 7 is a perspective view of a cannula according to a second embodiment;

FIG. 8 is a vertical cross-sectional view of an internal structure of an attachment portion and a junction portion provided in the cannula according to the second embodiment;

FIG. 9 is a diagram of an attachment portion according to a modification of the first embodiment and the second embodiment;

FIG. 10 is a perspective view of a cannula according to a third embodiment;

FIG. 11 is a vertical cross-sectional view of a structure of an attachment portion and a junction portion provided in the cannula according to the third embodiment;

FIG. 12 is a perspective view of the cannula in a state in which a first connection portion is rotated 180 degrees with respect to the junction portion from a state illustrated in FIG. 10; and

FIG. 13 is a side view of a junction portion according to a modification of the third embodiment.

### DESCRIPTION OF EMBODIMENTS

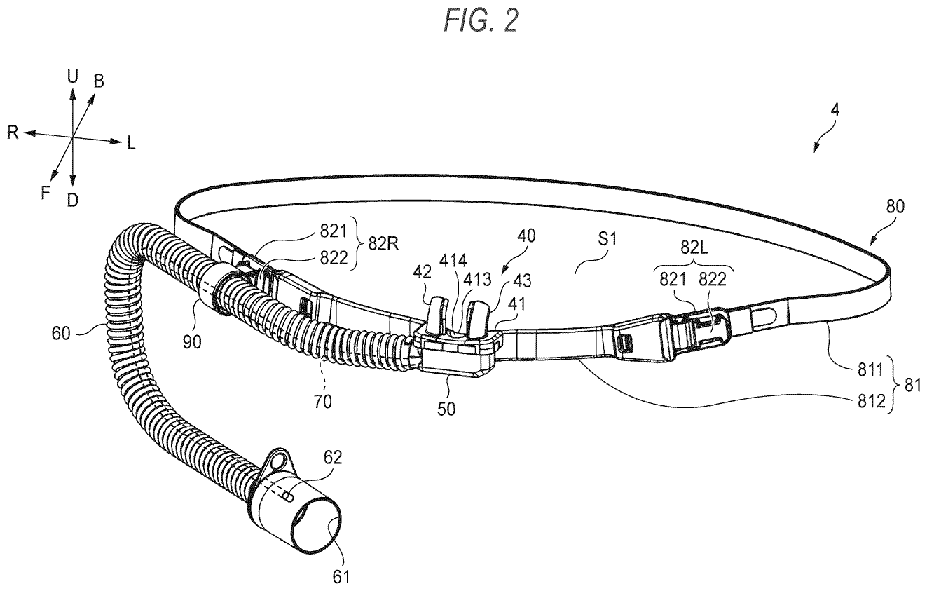

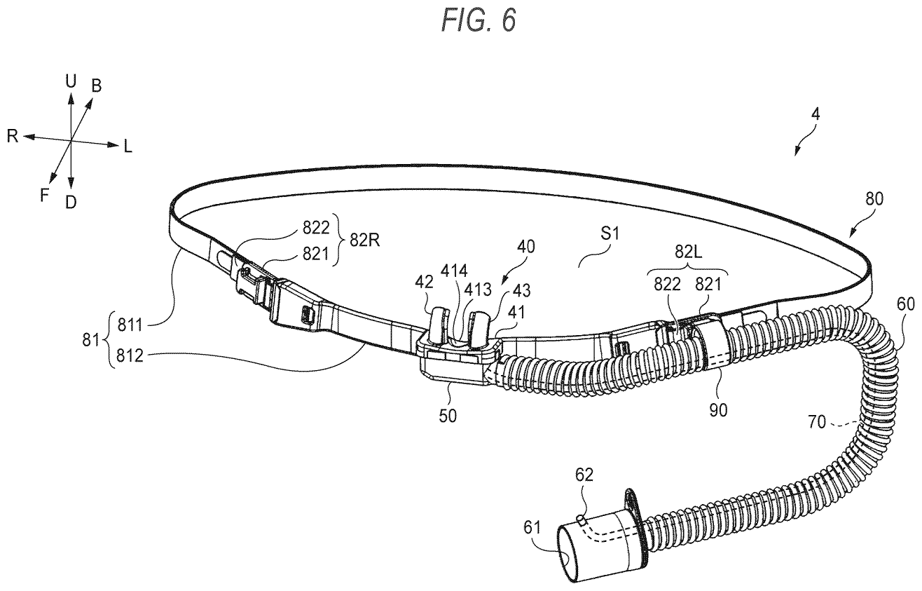

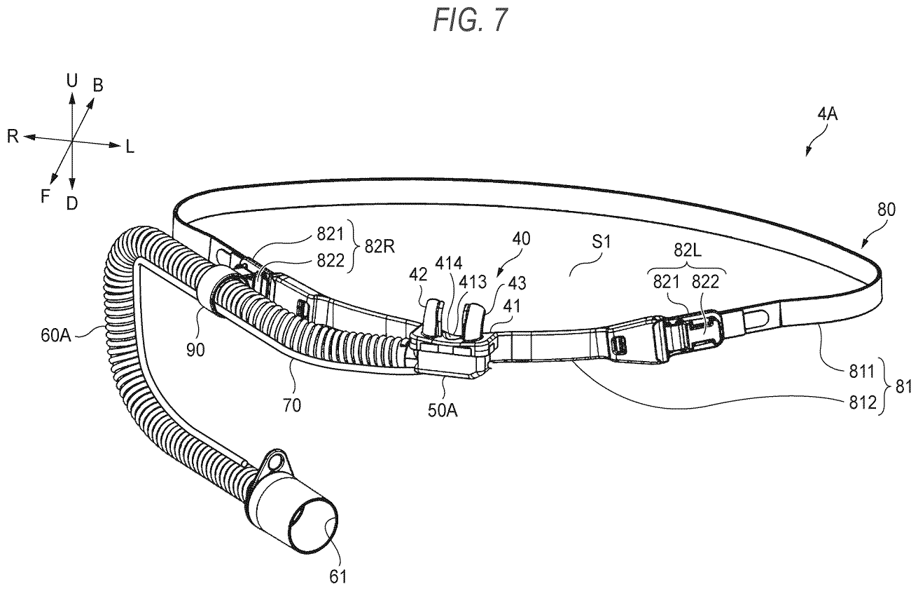

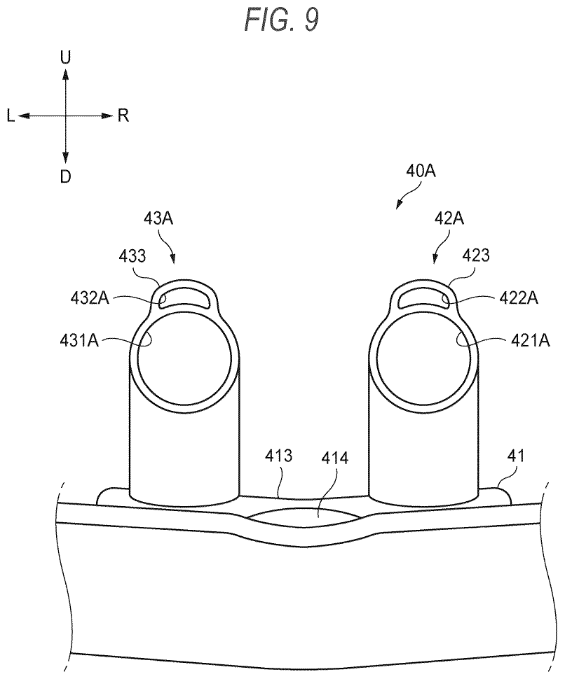

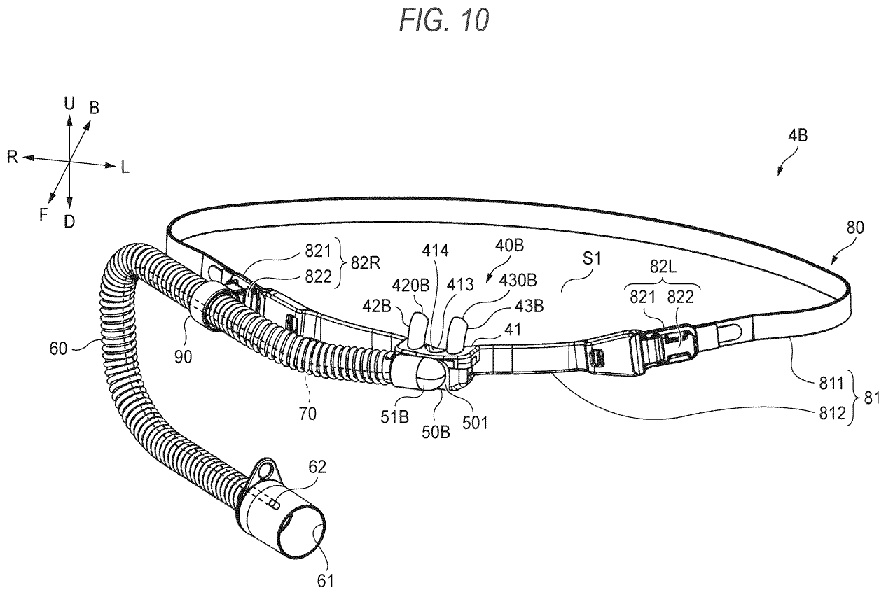

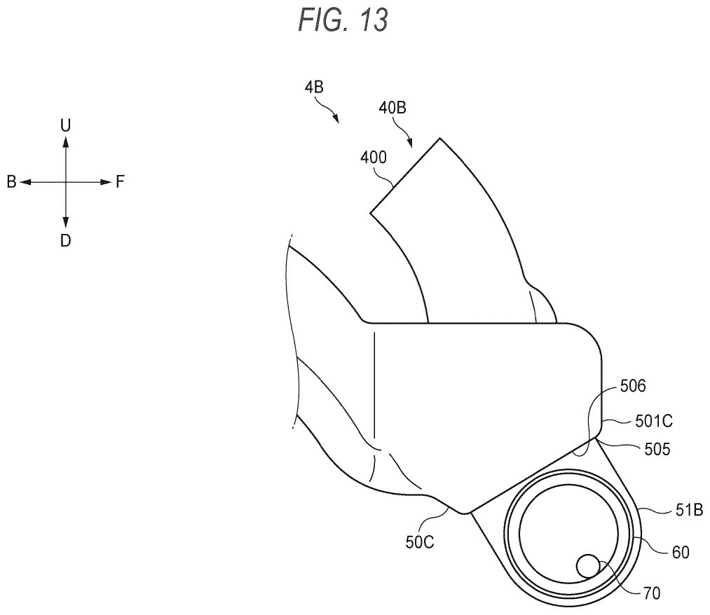

Hereinafter, an example of an embodiment of the present disclosure will be described below with reference to the drawings. In the description of the present embodiment, for convenience of description, terms “left-right direction”, “up-down direction”, and “front-back direction” may be described as appropriate. These directions are relative directions set for a cannula 4 illustrated in FIGS. 2 and 6, a cannula 4A illustrated in FIG. 7, an attachment portion 40A illustrated in FIG. 9, a cannula 4B illustrated in FIG. 10, and a junction portion 50C illustrated in FIG. 13. Here, the “left-right direction” is a direction including a “left direction” and a “right direction”. The “up-down direction” is a direction including an “up direction” and a “down direction”. The “front-back direction” is a direction including a “front direction” and a “back direction”. The front-back direction is a direction perpendicular to the left-right direction and the up-down direction. A reference character U illustrated in each drawing indicates the up direction. A reference character D indicates the down direction. A reference character F indicates the front direction. A reference character B indicates the back direction. A reference character L indicates the left direction. A reference character R indicates the right direction.

### First Embodiment

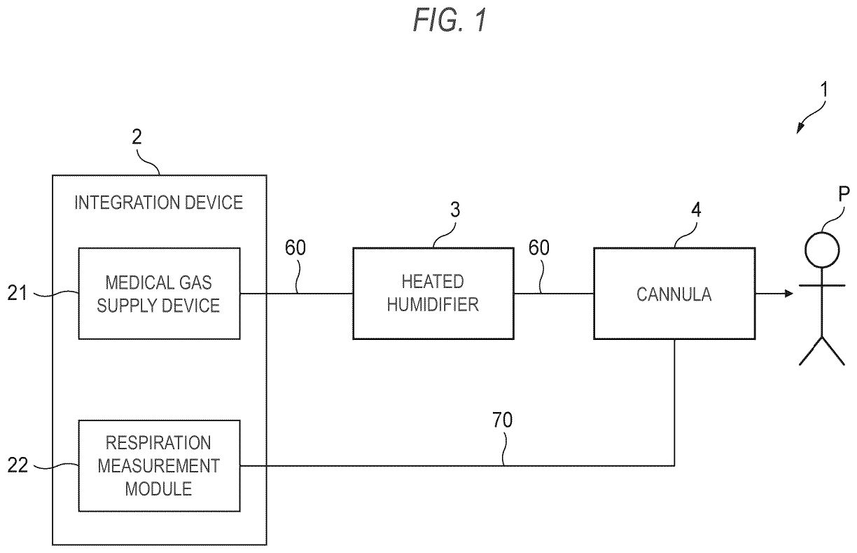

A medical system 1 of the present embodiment will be described with reference to FIGS. 1 and 2. FIG. 1 is a schematic diagram of the medical system 1 according to an embodiment of the present disclosure. The medical system 1 is used, for example, in a medical facility such as a hospital or the like. As illustrated in FIG. 1, the medical system 1 can include an integration device 2, a heated humidifier 3, and the cannula 4. The cannula 4 can include a medical gas conduit 60 and a measurement conduit 70. The cannula 4 is configured to be attached to a subject P.

The integration device 2 can include a medical gas supply device 21 and a respiration measurement module 22. The medical gas supply device 21 is connected to the heated humidifier 3 and the cannula 4, via the medical gas conduit 60. The medical gas supply device 21 is configured to generate a medical gas containing highly concentrated oxygen by using an adsorbent such as zeolite configured to adsorb nitrogen under high pressure and to release the adsorbed nitrogen under low pressure. The medical gas may contain components other than oxygen. Nitrogen released from the adsorbent under low pressure is discharged to the outside of the medical gas supply device 21. The medical gas supply device 21 is configured to supply the generated medical gas to the heated humidifier 3. In the present embodiment, an amount of the medical gas supplied from the medical gas supply device 21 to the heated humidifier 3 is, for example, 30 L/m to 60 L/m. In this way, in the present embodiment, the high flow medical gas is supplied from the medical gas supply device 21 toward the heated humidifier 3.

The respiration measurement module 22 is connected to the cannula 4 via the measurement conduit 70. The respiration measurement module 22 includes, for example, a piezoelectric element whose output changes depending on a pressure of exhalation or inhalation of the subject P and/or a flow rate of the exhalation and inhalation of the subject P. The respiration measurement module 22 is configured to calculate a respiratory rate of the subject P based on a pressure change and/or a flow rate change in the exhalation and inhalation of the subject P, to measure a respiratory condition of the subject P.

The heated humidifier 3 is configured to heat and to humidify the medical gas supplied from the medical gas supply device 21 to set, to an appropriate level, a temperature and a humidity of the medical gas. The heated humidifier 3 is configured to heat and to humidify the medical gas supplied from the medical gas supply device 21 by, for example, generating vapor inside the heated humidifier 3 and causing the medical gas to pass through the vapor.

Next, the cannula 4 will be described in detail with reference to FIGS. 2 to 6. In a case where the cannula 4 is attached to the subject P (see FIG. 1), the face of the subject P is positioned in a space S1 formed inside a fixing portion 80. Although not illustrated in FIG. 2, the medical gas supply device 21 (see FIG. 1) and the heated humidifier 3 (see FIG. 1) are connected directly or through other conduits to a first outlet portion 61 of the medical gas conduit 60, which is at a side opposite to an outlet connected to the cannula 4. The respiration measurement module 22 (see FIG. 1) is connected directly or through other conduits to a second outlet portion 62 of the measurement conduit 70, which is at a side opposite to the outlet connected to the cannula 4. As illustrated in FIG. 2, the cannula 4 can include an attachment portion 40, a junction portion 50, and the fixing portion 80.

The attachment portion 40 is a member for attaching the cannula 4 in nasal cavities of the subject P. The attachment portion 40 is made of, for example, a resin material such as silicone. The attachment portion 40 is soft, and hardness of the attachment portion 40 is Shore A30, for example. The attachment portion 40 can include a pedestal portion 41, a first attachment portion 42, and a second attachment portion 43.

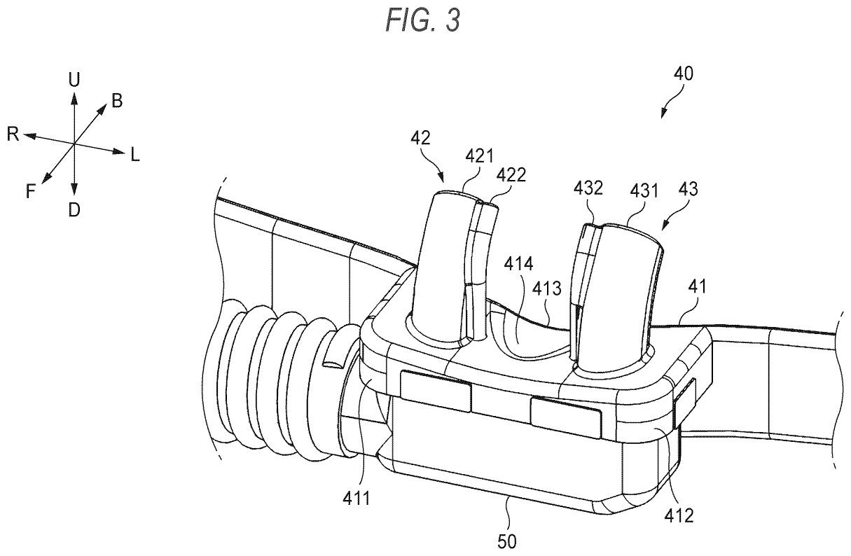

As illustrated in FIG. 3, the pedestal portion 41 has a substantially rectangular parallelepiped shape. A right front corner portion 411 and a left front corner portion 412 provided at both front ends of the pedestal portion 41 are rounded. However, the right front corner portion 411 and the left front corner portion 412 may be angular. A length, in a longitudinal direction (the left-right direction in FIG. 3), of the pedestal portion 41 is slightly larger than a length, in the longitudinal direction (the left-right direction in FIG. 3), of the junction portion 50. A length, in a lateral direction (the front-back direction in FIG. 3), of the pedestal portion 41 is slightly larger than a length, in the lateral direction (the front-back direction in FIG. 3), of the junction portion 50. A length, in a thickness direction (the up-down direction in FIG. 3), of the pedestal portion 41 is smaller than a length, in the thickness direction (the up-down direction in FIG. 3), of the junction portion 50. A central rear end portion 413 of the pedestal portion 41 is provided with a recessed portion 414. In a state where the subject P is attached with the cannula 4, a nasal columella of the subject P abuts against the recessed portion 414.

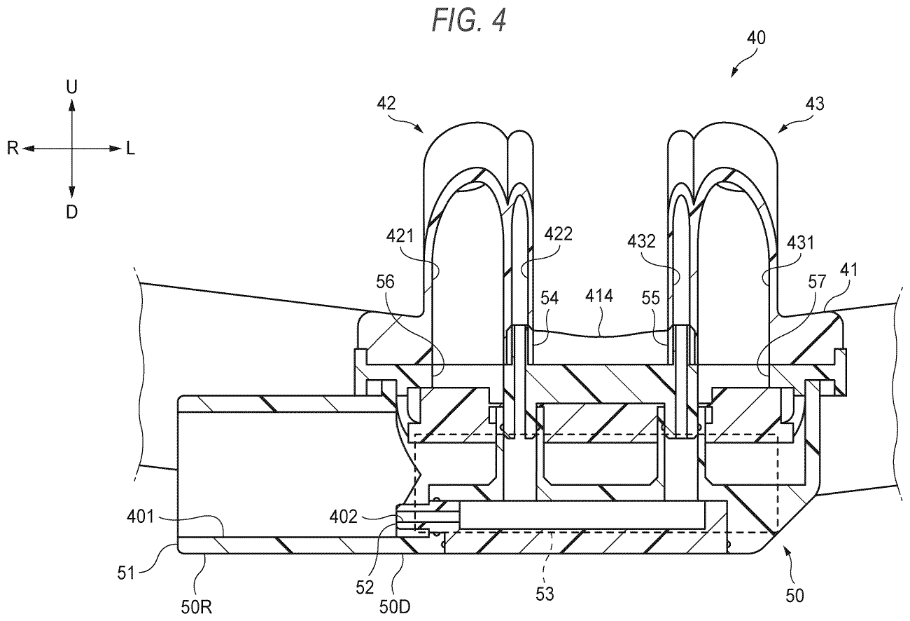

The first attachment portion 42 and the second attachment portion 43 are arranged at bilaterally symmetrical positions with respect to the recessed portion 414. The first attachment portion 42 is located on a right side of the recessed portion 414, and is configured to be attached in a right nasal cavity of the subject P. The second attachment portion 43 is located on a left side of the recessed portion 414, and is configured to be attached in a left nasal cavity of the subject P. The first attachment portion 42 and the second attachment portion 43 extend in the up direction from an upper surface of the pedestal portion 41. The first attachment portion 42 and the second attachment portion 43 are curved to extend backward as the first attachment portion 42 and the second attachment portion 43 extend upward. In other words, the first attachment portion 42 and the second attachment portion 43 have a substantially J shape. A bottom of the first attachment portion 42 and a bottom of the second attachment portion 43 are opened. A first mounting portion 54 (see FIG. 4) or a second mounting portion 55 (see FIG. 4), which will be described later, is mounted to the first attachment portion 42, and one of the first mounting portion 54 and the second mounting portion 55, which is not mounted to the first attachment portion 42, is mounted to the second attachment portion 43.

As illustrated in FIGS. 3 and 4, the first attachment portion 42 can include a first medical gas flow path 421 and a first measurement pipe 422. The second attachment portion 43 can include a second medical gas flow path 431 and a second measurement pipe 432. Therefore, each of the first attachment portion 42 and the second attachment portion 43 has a double lumen structure having a medical gas flow path and a measurement pipe.

The first medical gas flow path 421 and the second medical gas flow path 431 have, for example, a circular shape in cross section. An inner diameter of the first medical gas flow path 421 and an inner diameter of the second medical gas flow path 431 are, for example, 4 mm to 5 mm. The first measurement pipe 422 and the second measurement pipe 432 have, for example, a circular shape in cross section. An inner diameter of the first measurement pipe 422 and an inner diameter of the second measurement pipe 432 are, for example, 1 mm to 2 mm. Therefore, the inner diameter of the first medical gas flow path 421 is larger than the inner diameter of the first measurement pipe 422, and the inner diameter of the second medical gas flow path 431 is larger than the inner diameter of the second measurement pipe 432. Assuming that a sum of the inner diameter of the first medical gas flow path 421 and the inner diameter of the first measurement pipe 422 is set as an inner diameter of the first attachment portion 42, the inner diameter of the first attachment portion 42 is, for example, 5 mm to 7 mm. Assuming that a sum of the inner diameter of the second medical gas flow path 431 and the inner diameter of the second measurement pipe 432 is set as an inner diameter of the second attachment portion 43, the inner diameter of the second attachment portion 43 is, for example, 5 mm to 7 mm.

In the first attachment portion 42, the first medical gas flow path 421 and the first measurement pipe 422 are separate spaces (passages) that are independent of each other. The first medical gas flow path 421 is located farther from the recessed portion 414 than is the first measurement pipe 422, in the longitudinal direction of the attachment portion 40 (the left-right direction in FIGS. 3 and 4).

In the second attachment portion 43, the second medical gas flow path 431 and the second measurement pipe 432 are separate spaces (passages) that are independent of each other. The second medical gas flow path 431 is located farther from the recessed portion 414 than is the second measurement pipe 432, in the longitudinal direction of the attachment portion 40 (the left-right direction in FIGS. 3 and 4).

The junction portion 50 is configured to be connected to the attachment portion 40. As illustrated in FIG. 3, the junction portion 50 has a substantially rectangular parallelepiped shape. Corner portions of the junction portion 50 are chamfered and rounded. The junction portion 50 is made of, for example, a resin material such as polypropylene. Hardness of the junction portion 50 is Shore D70, for example. Therefore, the attachment portion 40 is softer than the junction portion 50.

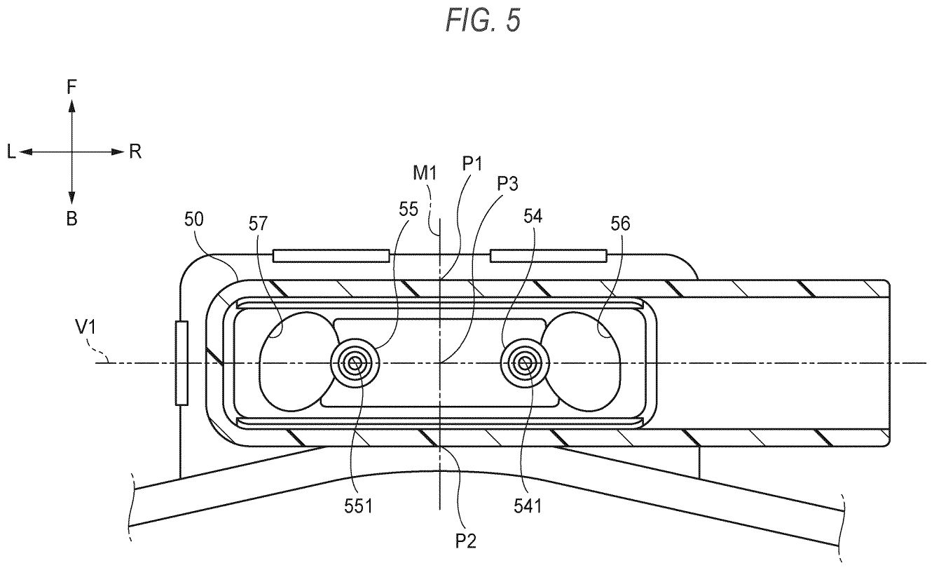

Here, an internal structure of the junction portion 50 will be described with reference to FIGS. 4 and 5. As illustrated in FIGS. 4 and 5, the junction portion 50 can include a first connection portion 51, a second connection portion 52, a branch portion 53, the first mounting portion 54, the second mounting portion 55, a first hole 56, and a second hole 57.

As illustrated in FIG. 4, the first connection portion 51 is provided at a right end portion 50R of the junction portion 50. The medical gas conduit 60 (see FIG. 2) is configured to be connected to the first connection portion 51. Therefore, the first connection portion 51 is provided to the junction portion 50 on a side of the medical gas supply device 21. An inner diameter of the first connection portion 51 is, for example, 10 mm to 12 mm. Therefore, the inner diameter of the first connection portion 51 is larger than the inner diameter of the first attachment portion 42 and the inner diameter of the second attachment portion 43. That is, the inner diameter of the first connection portion 51 is larger than an inner diameter of the attachment portion 40. The first connection portion 51 is provided with a single medical gas flow path 401 for supplying, to the subject P, the medical gas from the medical gas supply device 21. The medical gas flow path 401 has, for example, a circular shape in cross section.

The second connection portion 52 is provided at a lower end portion 50D of the junction portion 50. The second connection portion 52 is positioned inside the first connection portion 51. The measurement conduit 70 (see FIG. 2) is configured to be connected to the second connection portion 52. Therefore, the second connection portion 52 is provided to the junction portion 50 on a side of the respiration measurement module 22. A single measurement pipe 402 for measuring the respiratory condition of the subject P is provided in the second connection portion 52. The measurement pipe 402 has, for example, a circular shape in cross section. An inner diameter of the measurement pipe 402 is, for example, 1 mm to 2 mm. That is, the inner diameter of the measurement pipe 402 is equal to, for example, the inner diameter of the first measurement pipe 422 and the inner diameter of the second measurement pipe 432. The inner diameter of the measurement pipe 402 is smaller than an inner diameter of the medical gas flow path 401.

The branch portion 53 is provided on a left side of the first connection portion 51 and the second connection portion 52, in the junction portion 50. The branch portion 53 is configured to branch the medical gas flow path 401 and the measurement pipe 402 into the first attachment portion 42 and the second attachment portion 43. That is, the medical gas flow path 401 is branched, by the branch portion 53, into the first medical gas flow path 421 and the second medical gas flow path 431. The medical gas flow path 401 is continuously connected to the first medical gas flow path 421 and the second medical gas flow path 431. The measurement pipe 402 is branched, by the branch portion 53, into the first measurement pipe 422 and the second measurement pipe 432. The measurement pipe 402 is continuously connected to the first measurement pipe 422 and the second measurement pipe 432. In this way, the medical gas flow path 401 and the measurement pipe 402 are bifurcated by the branch portion 53.

The first mounting portion 54 is a member for mounting, to the junction portion 50, the first attachment portion 42 or the second attachment portion 43. As illustrated in FIGS. 4 and 5, the first mounting portion 54 has substantially convex shape. More specifically, the first mounting portion 54 has a cylindrical shape, and a top portion thereof has a substantially truncated cone shape. An outer diameter at the top portion of the first mounting portion 54 increases from an upper end portion toward a lower end portion. The first mounting portion 54 is male to the attachment portion 40 (the first attachment portion 42 and the second attachment portion 43), and the attachment portion 40 is female to the first mounting portion 54. For example, the first attachment portion 42 is configured to be inserted into the first mounting portion 54, to be mounted to the junction portion 50. A first circular hole 541 is provided in an upper end surface of the first mounting portion 54. The first mounting portion 54 is provided on a left side of the first hole 56.

The second mounting portion 55 is a member for mounting, to the junction portion 50, the first attachment portion 42 or the second attachment portion 43. The second mounting portion 55 has a substantially convex shape. More specifically, the second mounting portion 55 has a cylindrical shape, and a top portion thereof has a substantially truncated cone shape. An outer diameter at the top portion of the second mounting portion 55 increases from an upper end portion toward a lower end portion. The second mounting portion 55 is male to the attachment portion 40 (the first attachment portion 42 and the second attachment portion 43), and the attachment portion 40 is female to the second mounting portion 55. For example, the second attachment portion 43 is configured to be inserted into the second mounting portion 55, to be mounted to the junction portion 50. In this way, in the present embodiment, the junction portion 50 is male and the attachment portion 40 is female. A second circular hole 551 is provided in an upper end surface of the second mounting portion 55. The second mounting portion 55 is provided on a right side of the second hole 57.

As illustrated in FIG. 5, the first mounting portion 54 and the second mounting portion 55 are arranged line-symmetrical positions with respect to a middle line M1, which passes through middle points P1 and P2 of two long sides of the junction portion 50, that is, two sides extending in the longitudinal direction (the left-right direction in FIG. 5) of the junction portion 50. The middle line M1 passes through a middle point P3 of the first mounting portion 54 and the second mounting portion 55, and extends in a direction perpendicular to an imaginary line VI extending in a direction where the first mounting portion 54 and the second mounting portion 55 are arranged. The middle line M1 bisects a straight line connecting the first mounting portion 54 and the second mounting portion 55. The first mounting portion 54 and the second mounting portion 55 have a shape that is 180 degrees rotationally symmetrical about the middle point P3. The first mounting portion 54 and the second mounting portion 55 are bilaterally symmetrical with respect to the middle line M1. Therefore, in the present embodiment, the junction portion 50 can be mounted to the attachment portion 40 even in a case where the attachment portion 40 or the junction portion 50 is turned 180 degrees. In this way, in the present embodiment, a state where the junction portion 50 is connected to the attachment portion 40 can be switched between a first connection state and a second connection state, the first connection state in which the first mounting portion 54 is mounted to the first attachment portion 42 and the second mounting portion 55 is mounted to the second attachment portion 43, and the second connection state in which the second mounting portion 55 is mounted to the first attachment portion 42 and the first mounting portion 54 is mounted to the second attachment portion 43. Furthermore, an outer shape of an upper surface of a portion, other than the first connection portion 51, of the junction portion 50 is bilaterally symmetrical with respect to the middle line M1.

The first hole 56 has a substantially elliptical shape, when viewed from above. The first hole 56 is larger than the first circular hole 541. The second hole 57 has a substantially elliptical shape, when viewed from above. The second hole 57 is larger than the second circular hole 551. The first hole 56 and the second hole 57 are arranged in line-symmetrical positions with respect to the middle line M1.

Returning to FIG. 2, the medical gas conduit 60 will be described. The medical gas conduit 60 is configured to be connected to the first connection portion 51 (see FIG. 4). The medical gas conduit 60 is made of, for example, a soft resin material such as a vinyl chloride resin. Hardness of the medical gas conduit 60 is Shore A60, for example. Therefore, the medical gas conduit 60 is softer than the junction portion 50. As illustrated in FIG. 2, the medical gas conduit 60 is a corrugated tube. The medical gas conduit 60 is flexible. Therefore, the medical gas conduit 60 bends in a desired direction. In the present embodiment, the medical gas conduit 60 extends in the right direction from the junction portion 50, and is bent in a substantially U shape as an example in FIG. 2.

The measurement conduit 70 is configured to connected to the second connection portion 52 (see FIG. 4). Both the first connection portion 51 and the second connection portion 52 are provided on a right side of the junction portion 50. The measurement conduit 70 is located inside the medical gas conduit 60, and extends along the direction where the medical gas conduit 60 extends. The measurement conduit 70 is also made of a soft resin material and bends in a desired direction. Therefore, the measurement conduit 70 also extends in the right direction from the junction portion 50, and is bent in a U shape as an example in FIG. 2. Therefore, a connection direction of the medical gas conduit 60 to the first connection portion 51 and a connection direction of the measurement conduit 70 to the second connection portion 52 are identical. The measurement conduit 70 can be led out of the medical gas conduit 60 via the second outlet portion 62 provided in the medical gas conduit 60.

The fixing portion 80 can include a band-shaped portion 81 and conduit mounting portions 82. The band-shaped portion 81 extends in the left-right direction with a nose of the subject P (that is, the position of the recessed portion 414) as a reference. In a state illustrated in FIG. 2, the band-shaped portion 81 is annular. The band-shaped portion 81 can include a stretchable portion 811 and a non-stretchable portion 812.

The stretchable portion 811 is made of, for example, a stretchable material such as rubber. In the state illustrated in FIG. 2, the stretchable portion 811 is disposed behind the non-stretchable portion 812. The non-stretchable portion 812 is made of, for example, a synthetic resin such as plastic. The non-stretchable portion 812 is configured to be connected to the attachment portion 40.

The conduit mounting portions 82 are members for mounting the medical gas conduit 60 to the fixing portion 80. The respective conduit mounting portions 82 are provided on left and right sides of the band-shaped portion 81. In the present embodiment, the conduit mounting portion 82 provided on a right side of the attachment portion 40 is referred to as a right conduit mounting portion 82R, and the conduit mounting portion 82 provided on a left side thereof is referred to as a left conduit mounting portion 82L. Each of the conduit mounting portions 82 can include a plate-shaped portion 821 and a fastening portion 822. The plate-shaped portion 821 has a thin plate shape. The fastening portion 822 is provided on a front surface of the plate-shaped portion 821. The fastening portion 822 is substantially I-shaped, when viewed from the front. The fastening portion 822 can fasten the medical gas conduit 60 by, for example, inserting a holding member 90 that holds the medical gas conduit 60. In the state illustrated in FIG. 2, the holding member 90 that holds the medical gas conduit 60 is inserted into the fastening portion 822 of the right conduit mounting portion 82R.

On the other hand, in a case where the junction portion 50 is turned 180 degrees from the state illustrated in FIG. 2 and mounted to the attachment portion 40, a state illustrated in FIG. 6 is obtained. The state in which the junction portion 50 is connected to the attachment portion 40 in the state illustrated in FIG. 2 is the first connection state, and the state in which the junction portion 50 is connected to the attachment portion 40 in the state illustrated in FIG. 6 is the second connection state. In the state illustrated in FIG. 6, the holding member 90 that holds the medical gas conduit 60 is inserted into the fastening portion 822 of the left conduit mounting portion 82L. Therefore, in the state illustrated in FIG. 6, the medical gas conduit 60 and the measurement conduit 70 extend in the left direction from the junction portion 50, and are bent in a U shape. That is, the medical gas conduit 60 is connected to the first connection portion 51 such that the medical gas conduit 60 extends in the right direction or the left direction of the junction portion 50. In this way, the holding member 90 that holds the medical gas conduit 60 is inserted into the right conduit mounting portion 82R or the left conduit mounting portion 82L, and the conduit mounting portions 82 can mount the medical gas conduit 60 to the fixing portion 80.

Here, high flow oxygen therapy using the cannula 4 and measurement of the respiratory condition of the subject P performed during the high flow oxygen therapy will be described with reference to FIGS. 1 and 2. First, the cannula 4 is configured to attach the attachment portion 40 to the nasal cavities of the subject P, to be attached to the subject P. In this case, the face of the subject P is positioned in the space S1 formed inside the fixing portion 80, and the nasal columella of the subject P abuts the recessed portion 414. Next, the high flow medical gas is supplied from the medical gas supply device 21 toward the subject P attached with the cannula 4. After being heated and humidified by the heated humidifier 3, the medical gas is supplied to the subject P through the medical gas conduit 60, the medical gas flow path 401 (see FIG. 4), the first medical gas flow path 421 (see FIG. 4), and the second medical gas flow path 431 (see FIG. 4). The high flow oxygen therapy is performed in this way. On the other hand, during the high flow oxygen therapy, the respiration measurement module 22, which is connected to the cannula 4 via the measurement conduit 70, is configured to measure the respiratory condition of the subject P, based on a pressure change and/or a flow rate change in the exhalation and inhalation of the subject P. In this way, the respiratory condition of the subject P is measured while the high flow oxygen therapy is being performed on the subject P.

By the way, in a cannula used for high flow oxygen therapy, a high flow medical gas containing oxygen flows through a medical gas flow path provided in the cannula, and thus, a resistance applied to the medical gas flow path is relatively large. Therefore, for example, it is conceivable to provide a plurality of medical gas flow paths to reduce an amount of the medical gas flowing per one medical gas flow path. However, as the number of medical gas flow paths increases, the number of medical gas flow paths that come into contact with the outside air also increases, and thus, water droplets tend to be generated inside the medical gas flow paths through which a high-temperature and high-humidity medical gas flows. Furthermore, in the high flow oxygen therapy, it may be desirable to simultaneously supply a medical gas to a subject attached with a cannula and measure a respiratory condition of the subject. In this case, it is necessary to separately prepare a device for measuring the respiratory condition.

According to the cannula 4 having the above configuration, the first connection portion 51 is provided with the single medical gas flow path 401 for supplying the medical gas from the medical gas supply device 21 to the subject P, and the second connection portion 52 is provided with the single measurement pipe 402 for measuring the respiratory condition of the subject P. That is, since the cannula 4 has only one medical gas flow path 401 and one measurement pipe 402, the cannula 4 has a simple configuration. Since the cannula 4 has only one medical gas flow path 401, even in a case where the high flow medical gas flows through the medical gas flow path 401, water droplets are difficult to generate inside the medical gas flow path 401. Further, since the cannula 4 also includes the measurement pipe 402, the respiratory condition of the subject P can also be measured. In this way, the cannula 4 has a simple configuration, and is capable of making the medical gas flow path 401 difficult to generate water droplets inside and measuring the respiratory condition of the subject P while supplying the high flow medical gas.

According to the cannula 4 having the above configuration, the medical gas flow path 401 and the measurement pipe 402 are branched into the first attachment portion 42 and the second attachment portion 43 by the branch portion 53. The first attachment portion 42 can include the first medical gas flow path 421 and the first measurement pipe 422, and the second attachment portion 43 can include the second medical gas flow path 431 and the second measurement pipe 432. Since the first attachment portion 42 is attached to the right nasal cavity of the subject P, and the second attachment portion 43 is attached to the left nasal cavity of the subject P, according to the cannula 4, it is possible to effectively perform the high flow oxygen therapy and more easily know the respiratory condition of the subject P.

According to the cannula 4 having the above configuration, the medical gas conduit 60 is connected to the first connection portion 51 such that the medical gas conduit 60 extends in the right direction or the left direction of the junction portion 50. Therefore, a healthcare worker, the subject P, and the like can determine whether to extend the medical gas conduit 60 in the right direction of the junction portion 50 or in the left direction of the junction portion 50, according to a situation such as a posture of the subject P.

According to the cannula 4 having the above configuration, the state where the junction portion 50 is connected to the attachment portion 40 can be switched between the first connection state and the second connection state, the first connection state in which the first mounting portion 54 is mounted to the first attachment portion 42 and the second mounting portion 55 is mounted to the second attachment portion 43, and the second connection state in which the second mounting portion 55 is mounted to the first attachment portion 42 and the first mounting portion 54 is mounted to the second attachment portion 43. Therefore, for example, even in a case where the attachment portion 40 or the junction portion 50 is turned 180 degrees, the junction portion 50 can be connected to the attachment portion 40.

According to the cannula 4 having the above configuration, the first mounting portion 54 and the second mounting portion 55 have a shape that is 180 degrees rotationally symmetrical about the middle point P3. In the present embodiment, the pedestal portion 41 of the attachment portion 40 is disposed to cover the junction portion 50, and has a shape that is 180 degrees rotationally symmetrical about the middle point P3. Therefore, fitting portions (the mounting portions) of the junction portion 50 with respect to the attachment portion 40 have a shape that is 180 degrees rotationally symmetrical about the middle point P3, even in portions other than the first mounting portion 54 and the second mounting portion 55. Therefore, according to the cannula 4, the junction portion 50 can be reliably mounted to the attachment portion 40 even in a case where the junction portion 50 is mounted to the attachment portion 40 by turning the junction portion 50 in the left-right direction.

According to the cannula 4 having the above configuration, since the attachment portion 40 is softer than the junction portion 50, the cannula 4 can be firmly attached to the subject P while reducing a burden on the subject P attached with the cannula 4.

According to the cannula 4 having the above configuration, since the medical gas conduit 60 connected to the first connection portion 51 is softer than the junction portion 50, the medical gas conduit 60 can be easily bent in a desired direction. That is, it is possible to dispose the medical gas conduit 60 at an appropriate position according to a usage situation, and to reduce annoyance of the subject P.

According to the cannula 4 having the above configuration, the measurement conduit 70 connected to the second connection portion 52 is positioned inside the medical gas conduit 60 connected to the first connection portion 51. Therefore, according to the cannula 4 having the above configuration, compared with a case where the measurement conduit 70 is positioned outside the medical gas conduit 60, annoyance of the subject P can be reduced.

According to the cannula 4 having the above configuration, a connection direction of the medical gas conduit 60 to the first connection portion 51 and a connection direction of the measurement conduit 70 to the second connection portion 52 are identical. Therefore, according to the cannula 4 having the above configuration, for example, by disposing the medical gas conduit 60 and the measurement conduit 70 along each other, the medical gas conduit 60 and the measurement conduit 70 can be routed collectively.

According to the cannula 4 having the above configuration, since the inner diameter of the first connection portion 51 is larger than the inner diameter of the attachment portion 40, it is suitable for flowing the high flow medical gas.

According to the cannula 4 having the above configuration, the conduit mounting portions 82 for mounting the medical gas conduit 60 to the fixing portion 80 are provided on the left and right sides of the band-shaped portion 81. Therefore, according to the cannula 4 having the above configuration, the medical gas conduit 60 can be mounted at an appropriate position according to a posture of the subject P, for example.

### Second Embodiment

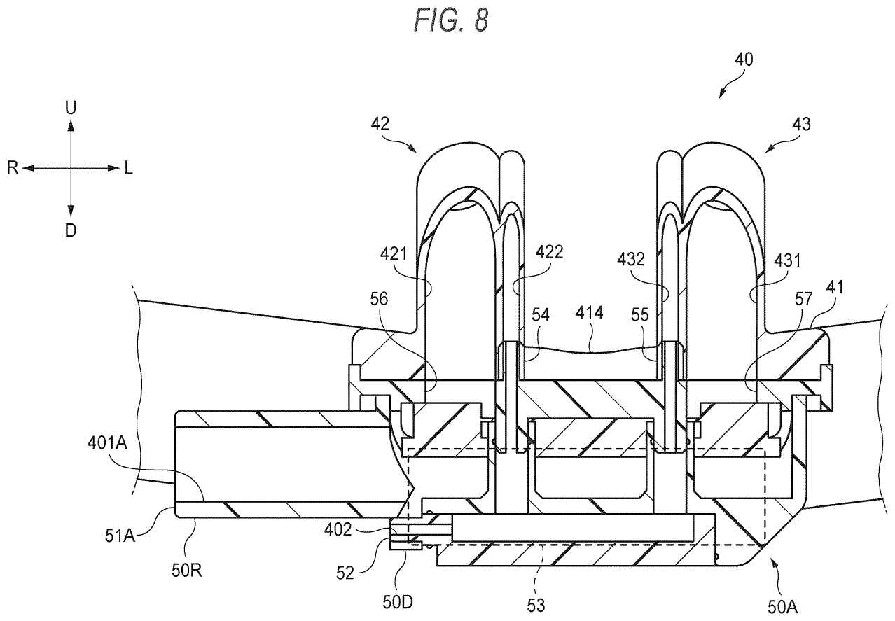

Next, the cannula 4A according to a second embodiment will be described with reference to FIGS. 7 and 8. In the present embodiment, the same reference character is attached to a portion same as that in the first embodiment, and a detailed description is omitted appropriately. FIG. 7 is a perspective view of the cannula 4A according to the second embodiment. FIG. 8 is a vertical cross-sectional view of an internal structure of the attachment portion 40 and a junction portion 50A provided in the cannula 4A according to the second embodiment. As illustrated in FIGS. 7 and 8, the cannula 4A is different from the cannula 4 according to the first embodiment in that the junction portion 50A is provided instead of the junction portion 50, the second connection portion 52 is positioned outside a first connection portion 51A, a medical gas conduit 60A is provided instead of the medical gas conduit 60, and the measurement conduit 70 is positioned outside the medical gas conduit 60A. The cannula 4A can include the attachment portion 40, the junction portion 50A, the medical gas conduit 60A, the measurement conduit 70, and the fixing portion 80.

Hardness of the junction portion 50A is Shore D70, for example. Therefore, the attachment portion 40 is softer than the junction portion 50A. As illustrated in FIG. 8, the junction portion 50A can include the first connection portion 51A, the second connection portion 52, the branch portion 53, the first mounting portion 54, the second mounting portion 55, the first hole 56, and the second hole 57. An inner diameter of the first connection portion 51A is smaller than the inner diameter of the first connection portion 51 according to the first embodiment, and the second connection portion 52 is positioned outside the first connection portion 51A. The inner diameter of the first connection portion 51A is larger than the inner diameter of the first attachment portion 42 and the inner diameter of the second attachment portion 43. That is, the inner diameter of the first connection portion 51A is larger than the inner diameter of the attachment portion 40. A medical gas flow path 401A having a circular shape in cross section is provided in the first connection portion 51A. An inner diameter of the medical gas flow path 401A is smaller than the inner diameter of the medical gas flow path 401 according to the first embodiment. Since the medical gas conduit 60A (see FIG. 7) is connected to the first connection portion 51A and the measurement conduit 70 (see FIG. 7) is connected to the second connection portion 52, the measurement conduit 70 is positioned outside the medical gas conduit 60A, as illustrated in FIG. 7.

The medical gas conduit 60A is substantially the same as or similar to the medical gas conduit 60, and is different from the medical gas conduit 60 in that the inner diameter of the medical gas conduit 60A is smaller than the inner diameter of the medical gas conduit 60 and that the second outlet portion 62 is not provided. Hardness of the medical gas conduit 60A is Shore A60, for example. Therefore, the medical gas conduit 60A is softer than the junction portion 50A.

As illustrated in FIG. 7, in the present embodiment, the holding member 90 that holds the medical gas conduit 60A is inserted into the fastening portion 822 of the right conduit mounting portion 82R. The medical gas conduit 60A provided in the cannula 4A extends in the right direction from the junction portion 50A, and is bent in a U shape.

The measurement conduit 70 provided in the cannula 4A extends along a direction where the medical gas conduit 60A extends. The measurement conduit 70 is held by a holding member 90 together with the medical gas conduit 60A. However, the measurement conduit 70 may not be held by the holding member 90. In this case, since the measurement conduit 70 can be disposed without depending on the medical gas conduit 60A, for example, the measurement conduit 70 can be extended in a direction different from the direction where the medical gas conduit 60A extends.

The cannula 4A according to the second embodiment can also achieve the same or similar effect as or to that of the cannula 4 according to the first embodiment.

According to the cannula 4A having the above configuration, the measurement conduit 70 connected to the second connection portion 52 is positioned outside the medical gas conduit 60A connected to the first connection portion 51A. That is, in the cannula 4A, the medical gas conduit 60A and the measurement conduit 70 are independent from each other, and thus, maintenance of the medical gas conduit 60A and the measurement conduit 70 can be performed separately. In this way, according to the cannula 4A, maintainability of the medical gas conduit 60A and the measurement conduit 70 is high.

### Modification of First Embodiment and Second Embodiment

Next, the attachment portion 40A according to a modification will be described with reference to FIG. 9. In the present modification, the same reference character is attached to a portion same as that in the first embodiment, and a detailed description is omitted appropriately. FIG. 9 is a diagram of the attachment portion 40A according to the modification. The attachment portion 40A is different from the attachment portion 40 according to the first embodiment in that the attachment portion 40A includes a first attachment portion 42A and a second attachment portion 43A instead of the first attachment portion 42 and the second attachment portion 43. Hardness of the attachment portion 40A is Shore A30, for example. Therefore, the attachment portion 40A is softer than the junction portion 50, 50A. In the present modification, the attachment portion 40A is female, and the junction portion 50, 50A is male.

As illustrated in FIG. 9, the attachment portion 40A can include the pedestal portion 41, the first attachment portion 42A, and the second attachment portion 43A. The first attachment portion 42A and the second attachment portion 43A are arranged at bilaterally symmetrical positions in the left-right direction with respect to the recessed portion 414. The first attachment portion 42A is located on the right side of the recessed portion 414, and is attached in the right nasal cavity of the subject P (see FIG. 1). The second attachment portion 43A is located on the left side of the recessed portion 414, and is attached in the left nasal cavity of the subject P. Same as or similar to the first attachment portion 42 and the second attachment portion 43 according to the first embodiment, the first attachment portion 42A and the second attachment portion 43A extend in the up direction from the upper surface of the pedestal portion 41 and have a substantially J shape. An inner diameter of the first attachment portion 42A and an inner diameter of the second attachment portion 43A are, for example, 5 mm to 7 mm. Therefore, the inner diameter of the first connection portion 51, 51A is larger than the inner diameter of the first attachment portion 42A and the inner diameter of the second attachment portion 43A. That is, the inner diameter of the first connection portion 51, 51A is larger than the inner diameter of the attachment portion 40A.

The first attachment portion 42A can include a first medical gas flow path 421A, a first measurement pipe 422A, and a first housing portion 423. Therefore, the first attachment portion 42A has a double lumen structure having a medical gas flow path and a measurement pipe. The first medical gas flow path 421A has, for example, a circular shape in cross section. The first measurement pipe 422A has, for example, a crescent shape in cross section, when viewed from the subject P attached with the cannula 4. The first medical gas flow path 421A is provided on the rear side (a subject P side) of the first measurement pipe 422A. However, the first medical gas flow path 421A may, for example, be provided on a front side of the first measurement pipe 422A, or may be provided on a right side or a left side of the first measurement pipe 422A. The first medical gas flow path 421A is larger than the first measurement pipe 422A.

The first housing portion 423 can include the first medical gas flow path 421A and the first measurement pipe 422A inside. Therefore, the first medical gas flow path 421A and the first measurement pipe 422A are provided inside the first housing portion 423.

The second attachment portion 43A can include a second medical gas flow path 431A, a second measurement pipe 432A, and a second housing portion 433. Therefore, the second attachment portion 43A has a double lumen structure having a medical gas flow path and a measurement pipe. The second medical gas flow path 431A has, for example, a circular shape in cross section. The second measurement pipe 432A has, for example, a crescent shape in cross section, when viewed from the subject P attached with the cannula 4. The second medical gas flow path 431A is provided on a rear side (the subject P side) of the second measurement pipe 432A. However, the second medical gas flow path 431A may, for example, be provided on a front side of the second measurement pipe 432A, or may be provided on a right side or a left side of the second measurement pipe 432A. The second medical gas flow path 431A is larger than the second measurement pipe 432A.

The second housing portion 433 includes the second medical gas flow path 431A and the second measurement pipe 432A inside. Therefore, the second medical gas flow path 431A and the second measurement pipe 432A are provided inside the second housing portion 433.

The cannula 4 including the attachment portion 40A according to the present modification can also achieve the same or similar effect as or to that of the cannula 4 including the attachment portion 40 according to the first embodiment.

### Third Embodiment

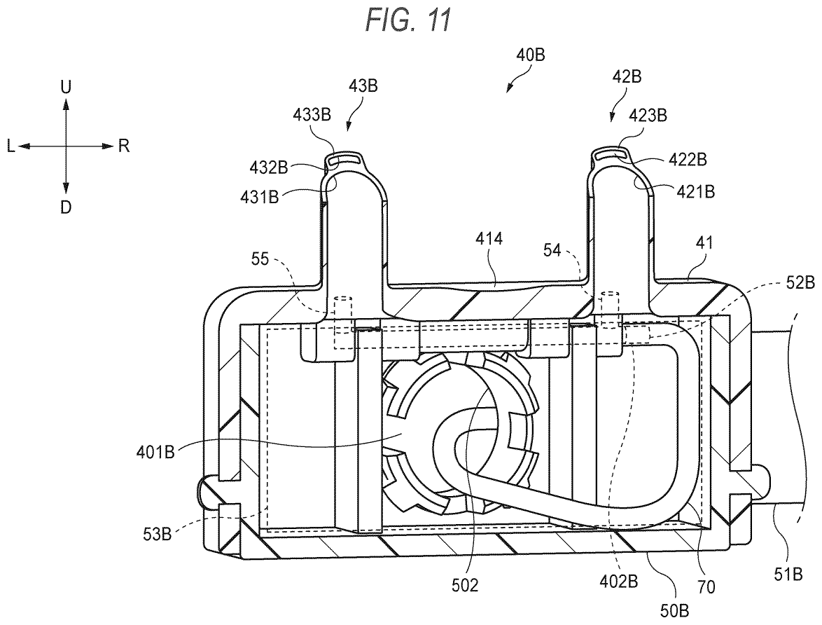

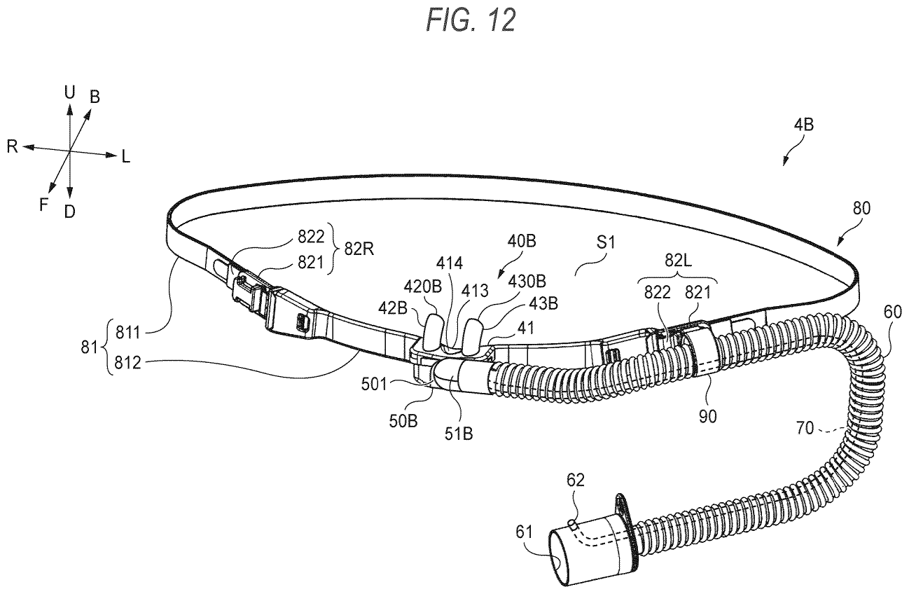

Next, the cannula 4B according to a third embodiment will be described with reference to FIGS. 10 and 12. In the present embodiment, the same reference character is attached to a portion same as that in the first embodiment, and a detailed description is omitted appropriately. FIG. 10 is a perspective view of the cannula 4B according to the third embodiment. FIG. 11 is a vertical cross-sectional view of a structure of an attachment portion 40B and a junction portion 50B provided in the cannula 4B according to the third embodiment. FIG. 12 is a perspective view of the cannula 4B in a state in which a first connection portion 51B is rotated 180 degrees with respect to the junction portion 50B from a state illustrated in FIG. 10. As illustrated in FIG. 10, the cannula 4B is different from the cannula 4 according to the first embodiment in that the attachment portion 40B is provided instead of the attachment portion 40, and that the junction portion 50B is provided instead of the junction portion 50. The cannula 4B can include the attachment portion 40B, the junction portion 50B, the medical gas conduit 60, the measurement conduit 70, and the fixing portion 80. Hardness of the attachment portion 40B is Shore A30, for example. Hardness of the junction portion 50B is Shore D70, for example. Therefore, the attachment portion 40B is softer than the junction portion 50B. Also in the present embodiment, the attachment portion 40B is female and the junction portion 50B is male.

As illustrated in FIG. 10, the attachment portion 40B can include the pedestal portion 41, the first attachment portion 42B, and the second attachment portion 43B. The first attachment portion 42B and the second attachment portion 43B are arranged at bilaterally symmetrical positions in the left-right direction with respect to the recessed portion 414. The first attachment portion 42B is located on the right side of the recessed portion 414, and is attached in the right nasal cavity of the subject P (see FIG. 1). The second attachment portion 43B is located on the left side of the recessed portion 414, and is attached in the left nasal cavity of the subject P. Same as or similar to the first attachment portion 42 and the second attachment portion 43 according to the first embodiment, the first attachment portion 42B and the second attachment portion 43B extend in the up direction from the upper surface of the pedestal portion 41 and have a substantially J shape. An inner diameter of the first attachment portion 42B and an inner diameter of the second attachment portion 43B are, for example, 5 mm to 7 mm.

As illustrated in FIG. 11, the first attachment portion 42B can include a first medical gas flow path 421B, a first measurement pipe 422B, and a first housing portion 423B. Therefore, the first attachment portion 42B has a double lumen structure having a medical gas flow path and a measurement pipe. The first medical gas flow path 421B has, for example, a circular shape in cross section. The first measurement pipe 422B has, for example, a crescent shape in cross section, when viewed from the subject P attached with the cannula 4B. The first medical gas flow path 421B is provided on a rear side (the subject P side) of the first measurement pipe 422B. However, the first medical gas flow path 421B may, for example, be provided on a front side of the first measurement pipe 422B, or may be provided on a right side or a left side of the first measurement pipe 422B. The first medical gas flow path 421B is larger than the first measurement pipe 422B.

The first housing portion 423B can include the first medical gas flow path 421B and the first measurement pipe 422B inside. Therefore, the first medical gas flow path 421B and the first measurement pipe 422B are provided inside the first housing portion 423B.

The second attachment portion 43B can include a second medical gas flow path 431B, a second measurement pipe 432B, and a second housing portion 433B. Therefore, the second attachment portion 43B has a double lumen structure having a medical gas flow path and a measurement pipe. The second medical gas flow path 431B has, for example, a circular shape in cross section. The second measurement pipe 432B has, for example, a crescent shape in cross section, when viewed from the subject P attached with the cannula 4B. The second medical gas flow path 431B is provided on a rear side (the subject P side) of the second measurement pipe 432B. However, the second medical gas flow path 431B may, for example, be provided on a front side of the second measurement pipe 432B, or may be provided on a right side or a left side of the second measurement pipe 432B. The second medical gas flow path 431B is larger than the second measurement pipe 432B.

The second housing portion 433B can include the second medical gas flow path 431B and the second measurement pipe 432B inside. Therefore, the second medical gas flow path 431B and the second measurement pipe 432B are provided inside the second housing portion 433B.

As illustrated in FIGS. 10 and 11, the junction portion 50B can include the first connection portion 51B, a second connection portion 52B, a branch portion 53B, the first mounting portion 54, and the second mounting portion 55. Since an upper portion of the junction portion 50B is open, the junction portion 50B does not have holes corresponding to the first hole 56 and the second hole 57 provided in the junction portion 50 according to the first embodiment. The first connection portion 51B has a substantially cylindrical shape. The first connection portion 51B is mounted to a mounting hole 502 formed in a front side surface 501 of the junction portion 50B. The mounting hole 502 is substantially circular. The first connection portion 51B may include a latch mechanism. In this case, in a case where the first connection portion 51B is rotated to a predetermined position after being inserted into the mounting hole 502, the first connection portion 51B is locked in the mounting hole 502 by the latch mechanism provided in the first connection portion 51B.

The first connection portion 51B is provided in the junction portion 50B in a direction (the front direction in FIG. 10) opposite to a direction (the back direction in FIG. 10) where portions (an upper portion 420B of the first attachment portion 42B and an upper portion 430B of the second attachment portion 43B), attached in the nasal cavities of the subject P, of the attachment portion 40B face. That is, the first connection portion 51B is provided on the front side surface 501 of the junction portion 50B. In this way, the first connection portion 51B is provided on a side (the front side in FIG. 10) opposite to the subject P, in a case where the subject P is attached with the cannula 4B. The first connection portion 51B is rotatable about the mounting hole 502 with respect to the junction portion 50B, in a state in which the first connection portion 51B is mounted in the mounting hole 502. That is, a structure of the junction portion 50B is a swivel structure. A range of the rotation of the first connection portion 51B when attached to the mounting hole 502 is from 0 degrees to 360 degrees with the mounting hole 502 as a reference. However, the range of the rotation of the first connection portion 51B is not limited thereto. The range of the rotation of the first connection portion 51B may be from 0 degrees to 180 degrees with the mounting hole 502 as a reference.

As illustrated in FIG. 10, the medical gas conduit 60 is connected to the first connection portion 51B. Therefore, the first connection portion 51B is provided to the junction portion 50B on the side of the medical gas supply device 21 (see FIG. 1). An inner diameter of the first connection portion 51B is, for example, 10 mm to 12 mm. Therefore, the inner diameter of the first connection portion 51B is larger than the inner diameter of the first attachment portion 42B and the inner diameter of the second attachment portion 43B. That is, the inner diameter of the first connection portion 51B is larger than an inner diameter of the attachment portion 40B. As illustrated in FIG. 11, the first connection portion 51B is provided with a single medical gas flow path 401B for supplying, to the subject P, the medical gas from the medical gas supply device 21. The medical gas flow path 401B has, for example, a circular shape in cross section.

As illustrated in FIG. 11, the second connection portion 52B is positioned inside the junction portion 50B. In the present embodiment, the second connection portion 52B is not positioned inside the first connection portion 51B. However, the second connection portion 52B may be positioned inside the first connection portion 51B. The measurement conduit 70 is connected to the second connection portion 52B. Therefore, the measurement conduit 70 is connected to the second connection portion 52B through the inside of the junction portion 50B. The measurement conduit 70 is positioned outside the junction portion 50B and positioned inside the medical gas conduit 60. Due to such a structure, in the present embodiment, the medical gas conduit 60 and the measurement conduit 70 can be extended in a desired direction simply by rotating the first connection portion 51B, without removing the measurement conduit 70. A single measurement pipe 402B for measuring the respiratory condition of the subject P is provided in the second connection portion 52. The measurement pipe 402B has, for example, a circular shape in cross section. An inner diameter of the measurement pipe 402B is, for example, 1 mm to 2 mm. The measurement pipe 402B extends in a longitudinal direction (the left-right direction in FIG. 11) of the junction portion 50B. The first mounting portion 54 and the second mounting portion 55 are provided in the measurement pipe 402B. The first mounting portion 54 and the second mounting portion 55 extend in the up direction in FIG. 11.

The branch portion 53B is configured to branch the medical gas flow path 401B and the measurement pipe 402B into the first attachment portion 42B and the second attachment portion 43B. That is, the medical gas flow path 401B is branched, by the branch portion 53B, into the first medical gas flow path 421B and the second medical gas flow path 431B. The measurement pipe 402B is branched, by the branch portion 53B, into the first measurement pipe 422B and the second measurement pipe 432B. The measurement pipe 402B is continuously connected to the first measurement pipe 422B and the second measurement pipe 432B. In this way, the medical gas flow path 401B and the measurement pipe 402B are bifurcated by the branch portion 53B.

The state illustrated in FIG. 10 becomes a state illustrated in FIG. 12, in a case where the medical gas conduit 60 is removed from the holding member 90 inserted into the fastening portion 822 of the right conduit mounting portion 82R, the first connection portion 51B is rotated 180 degrees with respect to the junction portion 50B about the mounting hole 502, and then the holding member 90 is inserted into the fastening portion 822 of the left conduit mounting portion 82L, and the medical gas conduit 60 is held by the holding member 90. In this way, the medical gas conduit 60 is connected to the first connection portion 51B such that the medical gas conduit 60 extends in the right direction or the left direction of the junction portion 50B. In the state illustrated in FIG. 10, the medical gas conduit 60 extends in the right direction of the junction portion 50B, but in the state illustrated in FIG. 12, the medical gas conduit 60 extends in the left direction of the junction portion 50B. That is, by rotating the first connection portion 51B 180 degrees with respect to the junction portion 50B about the mounting hole 502, the extending direction of the medical gas conduit 60 is changed to the right direction or the left direction.

The cannula 4B according to the third embodiment can also achieve the same or similar effect as or to that of the cannula 4 according to the first embodiment.

According to the cannula 4B having the above configuration, the first connection portion 51B rotates with respect to the junction portion 50B, so that the direction where the medical gas conduit 60 extends can be changed to the right direction or the left direction of the junction portion 50B. Therefore, a healthcare worker or the subject P can flexibly determine whether to set the direction where the medical gas conduit 60 extends as the right direction of the junction portion 50B or as the left direction of the junction portion 50B, according to a situation such as a posture of the subject P. According to the cannula 4B having the above configuration, the first connection portion 51B can be rotated without removing the measurement conduit 70 connected to the second connection portion 52B, and thus, the direction where the medical gas conduit 60 extends can be easily changed.

According to the cannula 4B having the above configuration, the first connection portion 51B is provided in the junction portion 50B in a direction (the front direction in FIG. 10) opposite to a direction (the back direction in FIG. 10) where portions (the upper portion 420B of the first attachment portion 42B and the upper portion 430B of the second attachment portion 43B), attached in the nasal cavities of the subject P, of the attachment portion 40B face. That is, the first connection portion 51B is provided on the front side surface 501 of the junction portion 50B. For example, in a case where the first connection portion is provided on a lower side of the junction portion, when such a cannula is attached to the subject P, the first connection portion is located near the mouth of the subject P, and thus, there is a concern that the subject P may be annoyed. However, in the cannula 4B, the first connection portion 51B is provided in the junction portion 50B in the direction opposite to the direction where the portion, attached to the nasal cavities of the subject P, of the attachment portion 40B face. Therefore, even if the cannula 4B is attached to the subject P, the first connection portion 51B is not located near the mouth of the subject P, and thus, it is possible to prevent the subject P from being annoyed.

### Modification of Third Embodiment

Next, the junction portion 50C according to a modification of the third embodiment will be described with reference to FIG. 13. In the present modification, the same reference character is attached to a portion same as that in the third embodiment, and a detailed description is omitted appropriately. FIG. 13 is a side view illustrating the junction portion 50C according to the present modification. As illustrated in FIG. 13, the junction portion 50C is different from the junction portion 50B according to the third embodiment in that a slope 505 is provided on a side (the front side in FIG. 13) opposite to the subject P when the cannula 4B is attached to the subject P (see FIG. 1). In the present modification, the attachment portion 40B is female, and the junction portion 50C is male. Hardness of the junction portion 50C is Shore D70, for example. Therefore, the attachment portion 40B is softer than the junction portion 50C. Therefore, the medical gas conduit 60 is softer than the junction portion 50C.

In the present modification, the slope 505 is continuous with a front side surface 501C of the junction portion 50C, and is provided below the front side surface 501C. Also in the present modification, the measurement conduit 70 is positioned inside the medical gas conduit 60. An internal structure of the junction portion 50C according to the present modification is substantially the same as or similar to the internal structure of the junction portion 50B according to the third embodiment. In the present modification, the measurement conduit 70 is positioned inside the medical gas conduit 60, outside the junction portion 50C.

The slope 505 is inclined such that a length of the junction portion 50C in a height direction (the up-down direction in FIG. 13) gradually decreases from the back to the front. The slope 505 is provided with the first connection portion 51B. In a case where the cannula 4B is attached to the subject P, a connection surface 506 between the junction portion 50C and the first connection portion 51B faces an opening surface 400 of the attachment portion 40B on the subject P side. The medical gas supplied from the medical gas supply device 21 is supplied to the subject P through the medical gas conduit 60, the junction portion 50C, and the attachment portion 40B, and the connection surface 506 faces the opening surface 400, so that a resistance to the medical gas inside the junction portion 50C and the attachment portion 40B is small. Therefore, the medical gas passing through the interior of the junction portion 50C and the attachment portion 40B is supplied to the subject P without being subjected to a large resistance.

The cannula 4B including the junction portion 50C according to the present modification can also achieve the same or similar effect as or to that of the cannula 4B including the junction portion 50B according to the third embodiment.

According to the cannula 4B including the junction portion 50C according to the present modification, in a case where the cannula 4B is attached to the subject P, the connection surface 506 between the junction portion 50C and the first connection portion 51B faces the opening surface 400 of the attachment portion 40B on the side of the subject P. Therefore, the medical gas passing through the interior of the junction portion 50C and the attachment portion 40B is supplied to the subject P without being subjected to a large resistance. Therefore, according to the cannula 4B having the junction portion 50C according to the present modification, the medical gas can be supplied to the subject P efficiently.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

In the above embodiments, the medical system 1 includes the integration device 2 including the medical gas supply device 21 and the respiration measurement module 22, the heated humidifier 3, and the cannula 4, 4A, 4B, but the present disclosure is not limited thereto. The medical system 1 may include, for example, the medical gas supply device 21, the respiration measurement module 22, the heated humidifier 3, and the cannula 4, 4A, 4B. That is, the medical gas supply device 21 and the respiration measurement module 22 may not be integrated into one device.

In the above embodiments, the attachment portion 40, 40A, 40B includes the first attachment portion 42, 42A, 42B and the second attachment portion 43, 43A, 43B, and may include only one of the first attachment portion 42, 42A, 42B and the second attachment portion 43, 43A, 43B. In this case, the attachment portion 40, 40A, 40B is mounted on either one of the right nasal cavity and the left nasal cavity of the subject P.

In the above embodiments, the first mounting portion 54 and the second mounting portion 55 have a shape that is 180 degrees rotationally symmetrical about the middle point P3, and may not have a shape that is 180 degrees rotationally symmetrical about the middle point P3. The outer shape of the upper surface of the portion, other than the first connection portion 51, 51A, 51B, of the junction portion 50, 50A, 50B, 50C has a shape that is 180 degrees rotationally symmetrical about the middle point P3, and may not have the shape that is 180 degrees rotationally symmetrical about the middle point P3.

In the above embodiments, the attachment portion 40, 40A, 40B is softer than the junction portion 50, 50A, 50B, 50C, and may have hardness same as that of the junction portion 50, 50A, 50B, 50C, and may be harder than the junction portion 50, 50A, 50B, 50C.

In the above embodiments, the medical gas conduit 60, 60A is softer than the junction portion 50, 50A, 50B, 50C, and may have hardness same as that of the junction portion 50, 50A, 50B, 50C, and may be harder than the junction portion 50, 50A, 50B, 50C.

In the above embodiments, the inner diameter of the first connection portion 51, 51A, 51B is larger than the inner diameter of the attachment portion 40, 40A, 40B, and may be the length same as the inner diameter of the attachment portion 40, 40A, 40B, and may be smaller than the inner diameter of the attachment portion 40, 40A, 40B.

In the above embodiments, the fixing portion 80 has two conduit mounting portions 82 (the right conduit mounting portion 82R and the left conduit mounting portion 82L), and may be provided with at least one conduit mounting portion 82 on each of the left side and the right side of the band-shaped portion 81, and may have three or more conduit mounting portions 82.

In the above first embodiment and second embodiment, the first medical gas flow path 421, the second medical gas flow path 431, the first measurement pipe 422, and the second measurement pipe 432 have a circular shape in cross section, and may have another shape such as a rectangular tube shape in cross section.

In the above modification of the first embodiment and second embodiment, the third embodiment, and the modification of the third embodiment, the first medical gas flow path 421A, 421B and the second medical gas flow path 431A, 431B have a circular shape in cross section, and may have another shape such as a rectangular tube shape in cross section. In the above modification of the first embodiment and second embodiment, the third embodiment, and the modification of the third embodiment, the first measurement pipe 422A, 422B and the second measurement pipe 432A, 432B have a crescent shape in cross section when viewed from the subject P attached with the cannula 4, 4A, 4B, and may have another shape such as a circular shape in cross section.

In the above first embodiment and second embodiment, the first medical gas flow path 421 is located farther from the recessed portion 414 than is the first measurement pipe 422 in the longitudinal direction of the attachment portion 40, and may be located closer to the recessed portion 414 than is the first measurement pipe 422, for example. The second medical gas flow path 431 is located farther from the recessed portion 414 than is the second measurement pipe 432 in the longitudinal direction of the attachment portion 40, and may be located closer to the recessed portion 414 than is the second measurement pipe 432, for example.

In the above embodiments, the first measurement pipe 422, 422A, 422B may be provided inside the tube forming the first medical gas flow path 421, 421A, 421B. The second measurement pipe 432, 432A, 432B may be provided inside the tube forming the second medical gas flow path 431, 431A, 431B.

In the above embodiments, the attachment portion 40, 40A, 40B is female and the junction portion 50, 50A, 50B, 50C is male, and the attachment portion 40, 40A, 40B may be male and the junction portion 50, 50A, 50B, 50C may be female. In the first embodiment, the second embodiment, and the modification of the first embodiment and second embodiment, even if the attachment portion 40, 40A is male and the junction portion 50, 50A is female and even if the attachment portion 40, 40A or the junction portion 50, 50A are turned 180 degrees, the attachment portion 40, 40A can be mounted to the junction portion 50, 50A. The attachment portion 40, 40A, 40B and the junction portion 50, 50A, 50B, 50C respectively include one male and one female mounting portion, and accordingly, the attachment portion 40, 40A, 40B may be mounted to the junction portion 50, 50A, 50B, 50C.

In the above embodiments, the male first mounting portion 54 and the male second mounting portion 55 are connected to the female attachment portion 40, 40A, 40B, so that spatial (passage) connection between the medical gas flow path 401, 401A, 401B, the first medical gas flow path 421, 421A, 421B, and the second medical gas flow path 431, 431A, 431B, and spatial (passage) connection between the measurement pipe 402, 402B, the first measurement pipe 422, 422A, 422B, and the second measurement pipe 432, 432A, 432B are achieved, and the present disclosure is not limited thereto. The spatial (passage) connection between the medical gas flow path 401, 401A, 401B, the first medical gas flow path 421, 421A, 421B, and the second medical gas flow path 431, 431A, 431B, and the spatial (passage) connection between the measurement pipe 402, 402B, the first measurement pipe 422, 422A, 422B, and the second measurement pipe 432, 432A, 432B may be achieved by connection at a portion other than the first mounting portion 54, the second mounting portion 55, and the attachment portion 40, 40A, 40B. For example, by fixing the attachment portion 40, 40A, 40B and the junction portion 50, 50A, 50B, 50C with an adhesive tape or the like, the medical gas flow path 401, 401A, 401B, the first medical gas flow path 421, 421A, 421B, and the second medical gas flow path 431, 431A, 431B, and the measurement pipe 402, 402B, the first measurement pipe 422, 422A, 422B, and the second measurement pipe 432, 432A, 432B are abutted against each other, and the above spatial (passage) connection may be achieved.

In the above embodiments, the junction portion 50, 50A, 50B, and 50C has a substantially rectangular parallelepiped shape, and may have another shape such as a truncated triangular pyramid shape or a truncated quadrangular pyramid shape.

In the above embodiment, the first mounting portion 54 and the second mounting portion 55 have a cylindrical shape, and may have another shape such as a square shape.

In the third embodiment and the modification of the third embodiment, the measurement conduit 70 is outside the junction portion 50B, 50C and inside the medical gas conduit 60, and may be outside the medical gas conduit 60.

In the third embodiment and the modification of the third embodiment, the first connection portion 51B is provided on the front side surface 501, 501C of the junction portion 50B, 50C, and may be provided on left and right side surfaces or the lower side surface of the junction portions 50B, 50C, for example.

As described above, the present specification discloses the following matters.

(1) A cannula used in high flow oxygen therapy, the cannula including:
an attachment portion configured to be attached in nasal cavities of a subject; and
a junction portion configured to be connected to the attachment portion,
in which a first connection portion is provided with a single medical gas flow path for supplying, to the subject, a medical gas from the medical gas supply device, the first connection portion being provided to the junction portion on a side of a medical gas supply device, and
a second connection portion is provided with a single measurement pipe for measuring a respiratory condition of the subject, the second connection portion being provided to the junction portion on a side of a respiration measurement module.

(2) The cannula according to the above described (1),
in which the attachment portion includes:
   a first attachment portion configured to be attached in a right nasal cavity of the subject; and
   a second attachment portion configured to be attached in a left nasal cavity of the subject,
the junction portion includes:
   a branch portion configured to branch the medical gas flow path into the first attachment portion and the second attachment portion, and configured to branch the measurement pipe into the first attachment portion and the second attachment portion,
the first attachment portion includes the medical gas flow path and the measurement pipe, and
the second attachment portion includes the medical gas flow path and the measurement pipe.

(3) The cannula according to the above described (1) or (2), further including:
a medical gas conduit configured to be connected to the first connection portion,
in which the medical gas conduit is connected to the first connection portion such that the medical gas conduit extends in a right direction or a left direction of the junction portion.

(4) The cannula according to the above described (3),
in which the first connection portion is configured to be rotated with respect to the junction portion to change, to the right direction or the left direction of the junction portion, a direction in which the medical gas conduit extends.

(5) The cannula according to the above described (4),
in which the first connection portion is provided in the junction portion in a direction opposite to a direction in which a portion, attached in the nasal cavities of the subject, of the attachment portion faces.

(6) The cannula according to the above described (5),
in which, in a state where the cannula is attached to the subject, a connection surface between the junction portion and the first connection portion faces an opening surface of the attachment portion on a side of the subject.

(7) The cannula according to the above described (2) or (3),
in which the junction portion includes a first mounting portion and a second mounting portion, and
a state where the junction portion is connected to the attachment portion is configured to be switched between a first connection state and a second connection state, the first connection state being a state where the first mounting portion is mounted to the first attachment portion and the second mounting portion is mounted to the second attachment portion, the second connection state being a state where the second mounting portion is mounted to the first attachment portion and the first mounting portion is mounted to the second attachment portion.

(8) The cannula according to the above described (7),
in which the first mounting portion and the second mounting portion have a shape that is 180 degrees rotationally symmetrical about a middle point between the first mounting portion and the second mounting portion.

(9) The cannula according to any one of the above described (1) to (8),
in which the attachment portion is softer than the junction portion.

(10) The cannula according to any one of the above described (1) to (9), further including:
a medical gas conduit configured to be connected to the first connection portion,
in which the medical gas conduit is softer than the junction portion.

(11) The cannula according to any one of the above described (1) to (10),
in which the second connection portion is located inside the first connection portion, and
a measurement conduit connected to the second connection portion is located inside a medical gas conduit connected to the first connection portion.

(12) The cannula according to any one of the above described (1) to (11),
in which the second connection portion is located outside the first connection portion, and
a measurement conduit connected to the second connection portion is located outside a medical gas conduit connected to the first connection portion.

(13) The cannula according to any one of the above described (1) to (12), further including:
a medical gas conduit configured to be connected to the first connection portion; and
a measurement conduit configured to be connected to the second connection portion,
in which a connection direction of the medical gas conduit with respect to the first connection portion, and a connection direction of the measurement conduit with respect to the second connection portion are identical.

(14) The cannula according to any one of the above described (1) to (13),
in which an inner diameter of the first connection portion is larger than an inner diameter of the attachment portion.

(15) The cannula according to any one of the above described (1) to (14), further including:
a fixing portion configured to fix the cannula to the subject,
in which the fixing portion includes:
   a band-shaped portion extending in a left-right direction with a nose of the subject as a reference;
   conduit mounting portions for mounting, to the fixing portion, a medical
gas conduit connected to the first connection portion, and the conduit mounting portions are provided on left and right sides of the band-shaped portion.

What is claimed is:

1. A cannula used in high flow oxygen therapy, the cannula comprising:
an attachment portion configured to be attached in nasal cavities of a subject; and
a junction portion configured to be connected to the attachment portion,
wherein a first connection portion is provided with a single medical gas flow path for supplying, to the subject, a medical gas from the medical gas supply device, the first connection portion being provided to the junction portion on a side of a medical gas supply device, and
a second connection portion is provided with a single measurement pipe for measuring a respiratory condition of the subject, the second connection portion being provided to the junction portion on a side of a respiration measurement module.

2. The cannula according to claim 1,
wherein the attachment portion includes:
a first attachment portion configured to be attached in a right nasal cavity of the subject; and
a second attachment portion configured to be attached in a left nasal cavity of the subject,
the junction portion includes:
a branch portion configured to branch the medical gas flow path into the first attachment portion and the second attachment portion, and configured to branch the measurement pipe into the first attachment portion and the second attachment portion,
the first attachment portion includes the medical gas flow path and the measurement pipe, and
the second attachment portion includes the medical gas flow path and the measurement pipe.

3. The cannula according to claim 1, further comprising:
a medical gas conduit configured to be connected to the first connection portion,
wherein the medical gas conduit is connected to the first connection portion such that the medical gas conduit extends in a right direction or a left direction of the junction portion.

4. The cannula according to claim 3,
wherein the first connection portion is configured to be rotated with respect to the junction portion to change, to the right direction or the left direction of the junction portion, a direction in which the medical gas conduit extends.

5. The cannula according to claim 4,
wherein the first connection portion is provided in the junction portion in a direction opposite to a direction in which a portion, attached in the nasal cavities of the subject, of the attachment portion faces.

6. The cannula according to claim 5,
wherein, in a state where the cannula is attached to the subject, a connection surface between the junction portion and the first connection portion faces an opening surface of the attachment portion on a side of the subject.

7. The cannula according to claim 2,
wherein the junction portion includes a first mounting portion and a second mounting portion, and
a state where the junction portion is connected to the attachment portion is configured to be switched between a first connection state and a second connection state, the first connection state being a state where the first mounting portion is mounted to the first attachment portion and the second mounting portion is mounted to the second attachment portion, the second connection state being a state where the second mounting portion is mounted to the first attachment portion and the first mounting portion is mounted to the second attachment portion.

8. The cannula according to claim 7,
wherein the first mounting portion and the second mounting portion have a shape that is 180 degrees rotationally symmetrical about a middle point between the first mounting portion and the second mounting portion.

9. The cannula according to claim 1,
wherein the attachment portion is softer than the junction portion.

10. The cannula according to claim 1, further comprising:
a medical gas conduit configured to be connected to the first connection portion,
wherein the medical gas conduit is softer than the junction portion.

11. The cannula according to claim 1,
wherein the second connection portion is located inside the first connection portion, and
a measurement conduit connected to the second connection portion is located inside a medical gas conduit connected to the first connection portion.

12. The cannula according to claim 1,
wherein the second connection portion is located outside the first connection portion, and
a measurement conduit connected to the second connection portion is located outside a medical gas conduit connected to the first connection portion.

13. The cannula according to claim 1, further comprising:
a medical gas conduit configured to be connected to the first connection portion; and
a measurement conduit configured to be connected to the second connection portion,
wherein a connection direction of the medical gas conduit with respect to the first connection portion, and a connection direction of the measurement conduit with respect to the second connection portion are identical.

14. The cannula according to claim 1,
wherein an inner diameter of the first connection portion is larger than an inner diameter of the attachment portion.

15. The cannula according to claim 1, further comprising:
a fixing portion configured to fix the cannula to the subject,
wherein the fixing portion includes:
a band-shaped portion extending in a left-right direction with a nose of the subject as a reference;
conduit mounting portions for mounting, to the fixing portion, a medical gas conduit connected to the first connection portion, and
the conduit mounting portions are provided on left and right sides of the band-shaped portion.
